(19) United States
(12) Patent Application Publication
(10) Pub. No.: US 2026/0062660 A1
(43) Pub. Date: Mar. 5, 2026

(54) MESH ROLLED SCAFFOLD AND ADVANCED BIOREACTOR

(71) Applicant: Board of Supervisors of Louisiana State University and Agricultural and Mechanical College, Baton Rouge, LA (US)

(72) Inventor: Kidong Park, Baton Rouge, LA (US)

(21) Appl. No.: 19/379,493

(22) Filed: Nov. 4, 2025

Related U.S. Application Data
(62) Division of application No. 17/278,538.
(60) Provisional application No. 62/734,367, filed on Sep. 21, 2018.

(51) Int. Cl. C12M 1/12; C12M 1/00; C12M 1/02; C12M 1/42

(52) U.S. Cl. CPC C12M 25/14; C12M 23/06; C12M 23/20; C12M 25/02; C12M 35/02; C12M 41/18

(57) ABSTRACT

The present invention provides mesh rolled scaffold devices and bioreactor systems that can provide a large surface-to-volume ratio for expanded cell culture. The mesh rolled scaffolds minimize shear stress on cultured cells and support sufficient and uniform mass transfer rates of gases and nutrients. The mesh rolled scaffolds can be connected to a media source via holders in bioreactor systems to support large-scale expansion and maintenance of cell cultures. The present invention also provides the bioreactor systems that can include dialyzers and heat exchangers to modify media and other fluids passing through the systems. The bioreactor systems include media and other fluid reservoirs that can support high stirring rates between about 100 and 10000 rpm, and the overall systems can be pressurized between about 1 and 10 atm to increase gas exchange rates.

### CROSS-REFERENCE TO RELATED APPLICATIONS

This application claims priority to U.S. Provisional Patent Application No. 62/734,367, filed Sep. 21, 2018, the contents of which are incorporated by reference herein in its entirety.

### BACKGROUND OF THE INVENTION

In laboratories, adherent cells are typically cultured with culture flasks having culture areas of between 25 and 175 cm². However, large-scale cell expansion often requires over hundreds or thousands of such culture flasks, which is impractical due to the amount of required labor and space. Roller bottles (Liu Y L et al., Biotechniques, 2003, 34(1):184-189) or multilayer planar vessels (U.S. Pat. No. 8,178,345) can be used to provide much larger growth areas of between about 1,000 and 10,000 cm². Using these alternatives to expand cells tends to be an easy and more direct translation from culture flasks, but they are still limited in their scale-up potential.

Currently, for large scale culture of adherent cells, a number of different platforms are available, such as microcarrier-based stirred bioreactors (Eibes G et al., Journal of biotechnology, 2010, 146(4):194-197; Hu A Y-C et al., Vaccine, 2008, 26(45):5736-5740; Lundgren B et al., Bioseparation and Bioprocessing: Biochromatography, Membrane Separations, Modeling, Validation, 1998, 165-222; Nam J H et al., Biotechnology progress, 2007, 23(3):652-660), packed-bed bioreactors (Looby D et al., Cytotechnology, 1988, 1(4):339-346), fluidized-bed bioreactors (Keller J et al., Advances in Bioprocess Engineering, 1994, 115-121), and hollow fiber bioreactors (Ku K et al., Biotechnology and Bioengineering, 1981, 23(1):79-95). Among these, the microcarrier-based stirred bioreactors are widely used to culture cells that cannot survive as single cells or cell aggregates. Anchorage dependent cells are grown on outer surfaces of suspended microcarriers, which are essentially solid microspheres. The microcarrier-based stirred bioreactors can support large capacity and massive quantities of anchorage dependent cells can be produced in a single run.

As the capacity of a bioreactor increases, the surface-to-volume ratio of the cell suspension decreases. More vigorous stirring and aeration are necessary to maintain mass transfer rate of nutrients and gases for larger numbers of cells (Xing Z et al., Biotechnology and bioengineering, 2009, 103(4):733-746). However, this increases hydrodynamic shear stress, which can produce adverse effects on cells, such as reduced proliferation, low viability, and uncontrolled differentiation of stem cells (Croughan M S et al., Biotechnology and bioengineering, 1987, 29(1):130-141; Gupta P et al., Cytotechnology, 2016, 68(1):45-59; Leung H W et al., Tissue Engineering Part C: Methods, 2010, 17(2):165-172; Ng Y-C et al., Biotechnology and bioengineering, 1996, 50(6):627-635; O'Connor K C et al., Biotechnology techniques, 1992, 6(4):323-328). The trade-off between the mass transfer rate and the hydrodynamic shear stress makes large-scale expansion of shear-sensitive cells unreliable and leads to time-consuming optimization of operating conditions on each expansion stage, as those factors are typically affected by the bioreactor's capacity.

One of the approaches to address this issue is to optimize configuration and geometry of stirred bioreactors and their impellers for maximum media mixing and minimum hydrodynamic shear stress. Numerous studies were able to make improvements to a certain degree, yet they could not overcome the fundamental limit imposed by the finite diffusion rate of gases and nutrients and the hydrodynamics (Trummer E et al., Biotechnology and bioengineering, 2006, 94(6):1033-1044; Odeleye A O O et al., Chemical engineering science, 2014, 111:299-312; Cioffi M et al., Journal of biomechanics, 2008, 41(14):2918-2925; Sucosky P et al., Biotechnology and bioengineering, 2004, 85(1):34-46; Santiago P A et al., Process biochemistry, 2011, 46(1):35-45; Grein T A et al., Process Biochemistry, 2016, 51(9):1109-1119). Another approach is to locally shield cells from the hydrodynamic shear stress. This approach includes macroporous microcarriers (Ng Y-C et al., Biotechnology and bioengineering, 1996, 50(6):627-635, Nilsson K et al., Nature Biotechnology, 1986, 4(11):989-990), fiber discs in packed-bed reactors (Meuwly F et al., Biotechnology and bioengineering, 2006, 93(4):791-800; Petti S A et al., Biotechnology progress, 1994, 10(5):548-550), and various encapsulation methods (Bauwens C et al., Biotechnology and Bioengineering, 2005, 90(4):452-461; Jing D et al., Cell transplantation, 2010, 19(11):1397-1412). Generally, in these techniques, cells are placed inside microstructures to be protected from the hydrodynamic shear stress (Martens D E et al., Cytotechnology, 1996, 21(1):45-59). However, such protection makes it difficult for nutrients and gases to be uniformly available to the cells, as some of them are located deep inside the protective microstructures (Preissmann A et al., Cytotechnology, 1997, 24(2):121-134). For the very same reason, harvesting the cells is very challenging.

Stirred bioreactors pose another problem, which is the inability to treat the media separately from the cells. In certain situations, it may be desirable to maintain the media under a set of conditions, such as at a temperature, pressure, humidity, or other environment that is not optimal for cell growth. Also, the media needs to be stirred or aerated, generating strong shear stress that is harmful to the cells. In another scenario, it may be desirable to remove certain components out of the media without removing a culture of cells for increased efficiency. However, currently used stirred bioreactors cannot treat or process the media without removing the cells that are immersed in the media.

Therefore, there is a need for improved devices and systems that are capable of large-scale culturing of adherent cells and implementing precise control of culture media. The present invention addresses this need.

### SUMMARY OF THE INVENTION

In one aspect, the present invention relates to mesh rolled scaffold device comprising: at least one substantially planar film having an upper surface, a lower surface, a length, a width, and a thickness; and at least one mesh netting having a length, a width, and a thickness; wherein the at least one film and the at least one mesh netting are rollable together into a cylindrical rolled scaffold having alternating film and mesh netting layers, and wherein the thickness of the at least one mesh netting maintains a space between each of the film layers.

In one embodiment, the at least one film further comprises circuitry electrically connected to electrodes on its upper surface, lower surface, or both. In one embodiment, the at least one mesh netting is selected from the group consisting of: reverse osmosis feed spacers, wire screens, and netting. In one embodiment, the at least one film, the at least one mesh netting, or both have a length of between about 10 cm and 1000 m, a width between about 1 cm and 1000 cm, and a thickness between about 0.01 m to 1 mm, such that a cylindrical rolled scaffold has a height between about 1 cm and 1000 cm, and a radius between about 0.5 cm and 5 m. In one embodiment, an edge of the at least one film and an edge of the at least one mesh netting are joined together by an adhesive, a weld, a clamp, or a sewn thread. In one embodiment, the at least one film, the at least one mesh netting, or both are provided with a surface area increasing physical modification selected from the group consisting of: fibers, bumps, ridges, pits, grooves, and channels. In one embodiment, the at least one film, the at least one mesh netting, or both are provided with a cell growth promoting or cell growth inhibiting surface treatment or patterns of cell growth promoting or cell growth inhibiting surface treatments. In one embodiment, the surface treatment is applied using a method selected from the group consisting of: electrospinning, electrospraying, spin coating, dip coating, chemical vapor deposition, chemical solution deposition, physical vapor deposition, liquid bath immersion, thermal imprinting, engraving, stamping, and microcontact printing.

In one embodiment, the device further comprises at least one adhesion layer rollable between the at least one film and the at least one mesh netting, wherein the at least one adhesion layer comprises a high porosity and a large internal surface area. In one embodiment, the at least one adhesion layer is selected from the group consisting of: non-woven fiber fabrics, woven fiber fabrics, papers, foam sheets, cleanroom wipes, and air filters. In one embodiment, the at least one film, the at least one mesh netting, the at least one adhesion layer, and combinations thereof have a length of between about 10 cm and 1000 m, a width between about 1 cm and 1000 cm, and a thickness between about 0.01 m to 1 mm, such that a cylindrical rolled scaffold has a height between about 1 cm and 1000 cm, and a radius between about 0.5 cm and 5 m. In one embodiment, an edge of the at least one film, an edge of the at least one mesh netting, an edge of the at least one adhesion layer, and combinations thereof are joined together by an adhesive, a weld, a clamp, or a sewn thread. In one embodiment, the at least one film, the at least one mesh netting, the at least one adhesion layer, and combinations thereof are provided with a surface area increasing physical modification selected from the group consisting of: fibers, bumps, ridges, pits, grooves, and channels. In one embodiment, the at least one film, the at least one mesh netting, the at least one adhesion layer, and combinations thereof are provided with a cell growth promoting or cell growth inhibiting surface treatment or patterns of cell growth promoting or cell growth inhibiting surface treatments. In one embodiment, the surface treatment is applied using a method selected from the group consisting of: electrospinning, electrospraying, spin coating, dip coating, chemical vapor deposition, chemical solution deposition, physical vapor deposition, liquid bath immersion, thermal imprinting, engraving, stamping, and microcontact printing.

In another aspect, the present invention relates to a bioreactor system, comprising: at least rolled scaffold; at least one cylindrical holder, each comprising a hollow casing sized to fit a rolled scaffold, at least one inlet port at a first end, and at least one outlet port at an opposite second end; at least one reservoir; tubing fluidically connecting the at least one reservoir to each of the cylindrical holders; and at least one pump connected to the tubing.

In one embodiment, the rolled scaffold is constructed from at least one substantially planar film and at least one mesh netting rolled into a cylindrical rolled scaffold having alternating layers of film and mesh netting. In one embodiment, the rolled scaffold is constructed from at least one substantially planar film, at least one mesh netting, and at least one adhesion layer rolled into a cylindrical rolled scaffold having alternating layers of film, mesh netting, and adhesion layers. In one embodiment, the rolled scaffold is constructed from at least one substantially planar film having a plurality of elongate spacers attached to the film rolled into a cylindrical rolled scaffold, such that the spacers maintain a space between the rolled film layers.

In one embodiment, the at least one reservoir is fluidically connected to one or more media sources, gas sources, chemical reagents, and combinations thereof. In one embodiment, the tubing comprises one or more access ports upstream from the cylindrical holders, downstream from the cylindrical holders, or both. In one embodiment, the tubing comprises one or more sensors upstream from the cylindrical holders, downstream from the cylindrical holders, or both. In one embodiment, the one or more sensors are selected from the group consisting of: temperature sensors, flow sensors, pH sensors, gas concentration sensors, glucose sensors, and analyte sensors.

In one embodiment, the tubing comprises one or more stopcocks or valves configured to stop or divert flow of fluid within the system. In one embodiment, the at least one rolled scaffold, each within a cylindrical holder, is connected to the at least one reservoir in series, in parallel, and combinations thereof. In one embodiment, the tubing further comprises a dialyzer configured to separate out components of fluid within the tubing and to introduce components into fluid within the tubing. In one embodiment, the tubing further comprises at least one heat exchanger configured to change the temperature of fluid within the tubing, such that the at least one reservoir is maintained at a temperature that is different from the temperature of the at least one rolled scaffold. In one embodiment, the at least one heat exchanger is positioned upstream from the at least one rolled scaffold, downstream from the at least one rolled scaffold, or both. In one embodiment, the at least one reservoir comprises a stirring impeller configured to rotate between 100 and 10000 rpm. In one embodiment, the system is pressurized between 1 atm and 10 atm. In one embodiment, the tubing further comprises at least one dialyzer and at least one heat exchanger.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1A is a schematic of an exemplary mesh rolled scaffold, wherein a film is rolled together with a mesh spacer to maintain a predetermined gap between each turn. FIG. 1B depicts a magnified view of the mesh component of an exemplary mesh rolled scaffold.

FIG. 2 depicts an exemplary diagram of a mesh rolled scaffold fabrication setup. A film and a mesh is rolled together to form a mesh rolled scaffold. “M” in the center of the mesh rolled scaffold denotes a motor. Two rollers are attached to shafts which can be rotated with a torque.

FIG. 3A and FIG. 3B each depict different magnified views of a prototype mesh rolled scaffold. The depicted predetermined gap is 450 μm between each layer.

FIG. 4 depicts an exemplary bioreactor system incorporating a rolled scaffold.

FIG. 5 depicts an exemplary bioreactor system configuration having a dialyzer. The culture medium passing through the rolled scaffold is dialyzed, removing a portion of the spent culture medium and receiving molecules from the dialysate. Pumps, sensors, and other components are omitted for clarity.

FIG. 6 depicts an exemplary bioreactor system configuration having a heat exchanger. The culture medium is maintained at a first temperature and is temperature-adjusted before being passed through the rolled scaffold. The heat exchanger is also compatible with other bioreactor systems, including the system depicted in FIG. 5. Pumps, sensors, and other components are omitted for clarity.

FIG. 7A and FIG. 7B depict a prototype mesh rolled scaffold. (FIG. 7A) The prototype mesh rolled scaffold has a cross-sectional diameter of about 6 cm and a length of about 30 cm. (FIG. 7B) A magnified view of a portion of the prototype mesh rolled scaffold depicts Chinese hamster ovary (CHO) cells growing on the substrate film; a part of the mesh spacer is visible.

FIG. 8A and FIG. 8B depict the results of experiments culturing CHO cells on a prototype mesh rolled scaffold. FIG. 8A is a graph showing the oxygen consumption rates of CHO cells cultured in prototype mesh rolled scaffolds that are either coated with poly-L-lysine (PLL) or exposed to growth media before seeding. The number at the end of the graph shows the number of the harvested CHO cells. FIG. 8B is a graph showing oxygen consumption rates of CHO cells cultured in prototype mesh rolled scaffolds that are exposed to growth media before seeding. The number in the graph shows the number of the harvested CHO cells. The temperature was decreased to 31° C. at 96 hours to simulate the phase production.

### DETAILED DESCRIPTION

The present invention provides mesh rolled scaffold devices and bioreactor systems that can provide a large surface-to-volume ratio for expanded cell culture. The mesh rolled scaffolds minimize shear stress on cultured cells and support sufficient and uniform mass transfer rates of gases and nutrients. The mesh rolled scaffolds can be connected to a media source via holders in bioreactor systems to support large-scale expansion and maintenance of cell cultures. The present invention also provides bioreactor systems that can include dialyzers and heat exchangers to modify media and other fluids passing through the systems. The bioreactor systems described herein are compatible with any rolled scaffold, including mesh rolled scaffolds as well as rolled scaffolds using spacers formed by molds or ultraviolet light curing (UV rolled scaffolds).

The present invention also provides bioreactor systems that can culture adherent cells on monolayers in a large scale. In one embodiment, the bioreactor systems can be used to remove byproducts produced by the cultured cells. In one embodiment, the bioreactor systems can be used with a heat exchanger to maintain a rolled scaffold, a media reservoir, a dialysate reservoir, and combinations thereof at different temperatures. In one embodiment, the bioreactor systems can support pressurization between about 1 and 10 atm and stirring between about 100 and 10000 rpm. For example, oxygenated culture media with proper nutrients can be flowed through the rolled scaffold and the bioreactor systems, including pumping units, gas supply systems, and mixing units, can be implemented to maintain culture media with appropriate levels of nutrients, oxygen, carbon dioxide, byproduct, temperature, and pH, and to pump the culture media into the rolled scaffolds.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiation associated proteins in that cell. When a cell is said to be “differentiating,” as that term is used herein, the cell is in the process of being differentiated.

“Differentiation medium” is used herein to refer to a cell growth medium comprising an additive or a lack of an additive such that a stem cell, adipose derived adult stromal cell or other such progenitor cell, that is not fully differentiated when incubated in the medium, develops into a cell with some or all of the characteristics of a differentiated cell.

The term “derived from” is used herein to mean to originate from a specified source.

“Expandability” is used herein to refer to the capacity of a cell to proliferate, for example, to expand in number or in the case of a cell population to undergo population doublings.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein “growth factors” is intended the following non-limiting factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), transforming growth factor (TGF-beta), hepatocyte growth factor (HGF), and bone morphogenetic protein at concentrations of between picogram/ml to milligram/ml levels.

As used herein, the term “growth medium” is meant to refer to a culture medium that promotes growth of cells. A growth medium will generally contain animal serum. In some instances, the growth medium may not contain animal serum.

An “isolated cell” refers to a cell which has been separated from other components and/or cells which naturally accompany the isolated cell in a tissue or mammal.

As used herein, the term “multipotential” or “multipotentiality” is meant to refer to the capability of a stem cell to differentiate into more than one type of cell.

As used herein, a “pluripotent cell” defines a less differentiated cell that can give rise to at least two distinct (genotypically and/or phenotypically) further differentiated progeny cells.

The terms “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew itself or to produce progeny cells which will differentiate into the desired cell type. Unlike pluripotent stem cells, lineage-committed progenitor cells are generally considered to be incapable of giving rise to numerous cell types that phenotypically differ from each other. Instead, progenitor cells give rise to one or possibly two lineage-committed cell types.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of 3H-thymidine into the cell, and the like.

“Progression of or through the cell cycle” is used herein to refer to the process by which a cell prepares for and/or enters mitosis and/or meiosis. Progression through the cell cycle includes progression through the G1 phase, the S phase, the G2 phase, and the M-phase.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, “tissue engineering” refers to the process of generating tissues ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

### Mesh Rolled Scaffold

The mesh rolled scaffold is highly unique in that it can be fabricated in a cost-effective manner with a microarchitecture engineered for optimal transport of oxygen and nutrients, while it can achieve higher culture capacity than other culture platforms. Unlike stirred bioreactors for suspension culture, which rely on diffusion and turbulent flow for mass transport, the mesh rolled scaffold-based cell biomanufacturing platform transports nutrients and oxygen via convection and laminar flow with much higher efficiency, so that hydrodynamic shear stress is drastically reduced compared to stirred bioreactors. As the geometry of mesh rolled scaffolds is fully defined, hydrodynamic shear stress and mass transfer rate of nutrients and oxygen are highly uniform and can be precisely controlled, substantially increasing uniformity and reliability of biomanufacturing of therapeutic cells, including stem cells from various sources, protein therapeutics, antibodies, and any other biomolecules produced by cells. Furthermore, the microenvironment of the rolled scaffold is independent of the culture capacity, as the culture capacity can be increased by increasing the diameter and/or the length of the mesh rolled scaffold.

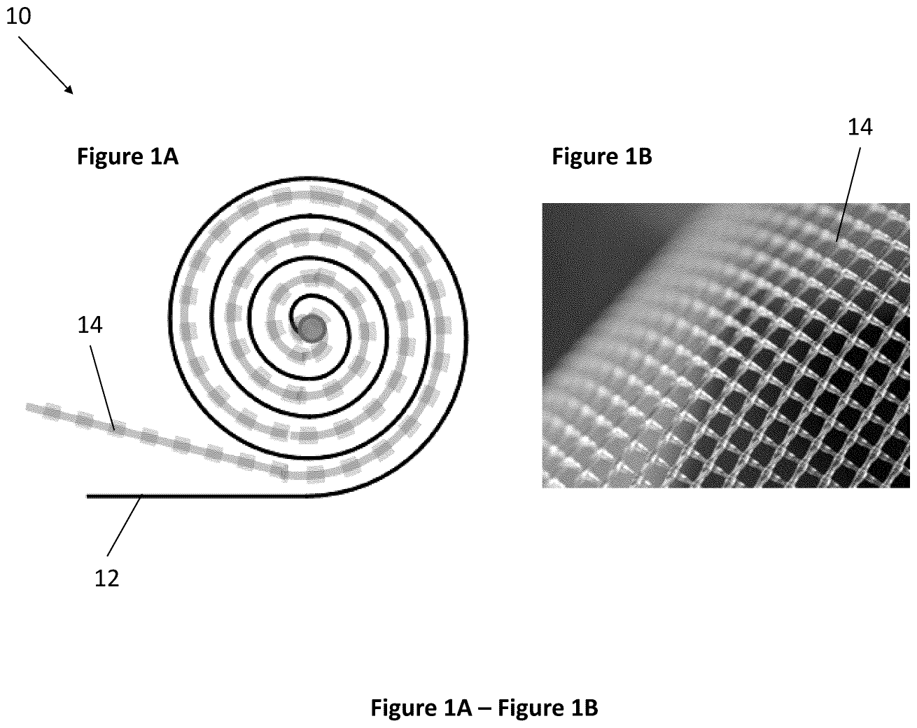

Referring now to FIG. 1A, an exemplary mesh rolled scaffold device 10 is depicted. Device 10 comprises film 12 and mesh spacer 14 rolled together. Film 12 has a substantially planar shape having a length, a width, and a thickness of any suitable size. For example, film 12 can have a length between about 10 cm and 1000 m or more, a width between about 1 to 1000 cm or more, and a thickness between about 0.01 to 1 mm or more. In some embodiments, film 12 can be augmented with circuitry, electrodes, magnets, diodes, and the like, such that film 12 can be electrified to support electroporation, a magnetic field, electric stimulation, impedance measurement, and the like. In some embodiments, film 12 can be modified to increase surface area, such as by adding one or more fibers, bumps, ridges, pits, grooves, channels, and the like.

Mesh spacer 14 is a netting-like structure comprising a pattern of overlaid filaments, as shown in FIG. 1B. While the depicted mesh spacer 14 comprises a pattern of evenly spaced filaments aligned perpendicular to each other, it should be understood that mesh spacer 14 can be constructed from a pattern of filaments having any suitable spacing and aligned at any suitable angle. The filaments of mesh spacer 14 can have any suitable cross-sectional width, such as a width between about 0.01 and 1 mm. The filaments of mesh spacer 14 can have any suitable cross-sectional shape, such as a square, rectangle, trapezoid, hexagon, triangle, circle segment, ovoid segment, and the like. In some embodiments, mesh spacer 14 can be adapted from a commonly available source, including but not limited to reverse osmosis feed spacers, wire screens, and netting.

In some embodiments, device 10 further comprises one or more adhesion layers positioned between each layer of film 12 and mesh spacer 14 (not pictured). The one or more adhesion layers has a flexible construction that enables it to be rolled within device 10. In some embodiments, the one or more adhesion layers have high porosity and a large internal surface area, wherein the high internal surface area accommodates cell adhesion for a large cell culture population and the high porosity permits the flow of fluid and the transfer of gases and nutrients. The one or more adhesion layers thereby can be constructed from any suitable material, including polymers and biologically derived components (e.g., extracellular matrix, collagen, fibrin, keratin, and the like). In some embodiments, the one or more adhesion layers can be adapted from a commonly available source, including but not limited to non-woven fiber fabrics, woven fiber fabrics, air filters, papers (such as filter paper), foam sheets, and cleanroom wipes.

The one or more films 12, mesh spacers 14, and adhesion layers can be joined to each other at an edge by any suitable attachment means, including but not limited to an adhesive, a weld, a clamp, a sewn thread, and the like.

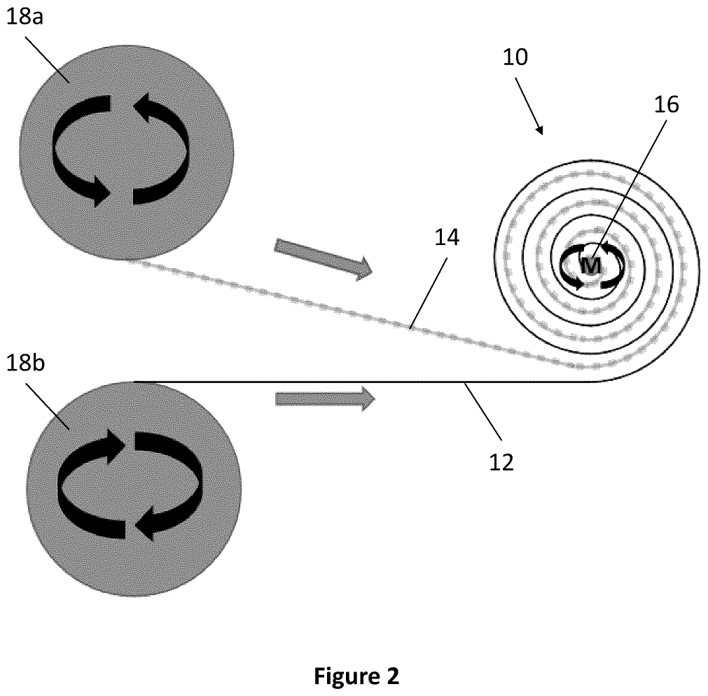

Referring now to FIG. 2, an exemplary fabrication setup of device 10 is depicted. A length of mesh spacer 14 can be wrapped around a first spool 18a, and a length of film 12 can be wrapped around a second spool 18b. First spool 18a and second spool 18b can each present a free edge of mesh spacer 14 and film 12, respectively, which can be attached to each other by any suitable attachment means described above, or directly attached to a spindle 16. In various embodiments, one or more additional spools are provided to incorporate one or more films 12, one or more mesh spacers 14, one or more adhesion layers, and combinations thereof into a device 10.

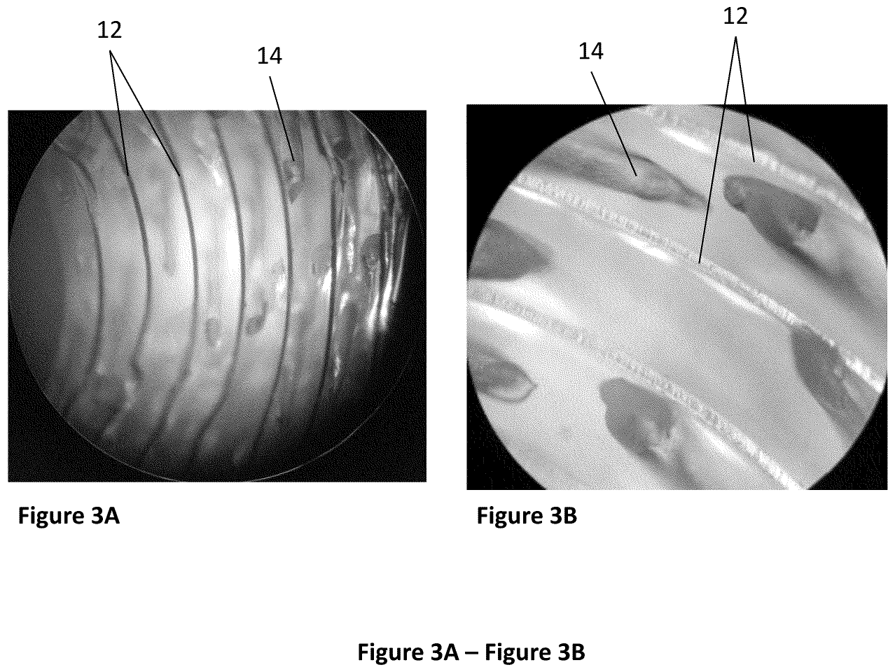

Fabrication can be achieved by using a spool 18 for each of the films 12 and mesh spacers 14, wherein a free edge of each of the films 12 and mesh spacers 14 are attached to a spindle 16. Spindle 16 can be mounted to a rotating drive and rotated, either manually or by a motor, to roll film 12 and mesh spacer 14 into a cylindrical shape to form device 10. After rolling, spindle 16 can be retained as part of device 10 or removed from device 10. As shown in FIG. 3A and FIG. 3B, rolling mesh spacer 14 and film 14 together forms alternating layers within device 10. The diameter of the filaments of mesh spacer 14 thereby maintains a constant separation between each layer of film 12, the width of film 12 becomes a height of device 10, and the rolled length of film 12 can be described as a radius or diameter of device 10. For example, device 10 can have a height between about 1 and 1000 cm or more, and a radius between about 0.5 cm to 5 m or more.

Film 12, mesh spacer 14, and the adhesion layers can each be constructed from any suitably flexible material, such as a plastic, a polymer, a paper, or a metal. In some embodiments, the material can be any suitable material that can support the growth of adherent cells. For example, the material can be selected from a polymer, including but not limited to: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO), polyorthoesters, and the like. In one embodiment, the material is polyethylene terephthalate (PET). In some embodiments, the material is capable of withstanding common sterilization techniques, such as autoclaving, gamma ray sterilization, electron beam sterilization, and the application of any sterilizing gas or solution such as ethylene oxide, chlorine dioxide, and hydrogen peroxide.

In some embodiments, the one or more films 12, mesh spacers 14, and adhesion layers can be physically modified to increase available surface area. The physical modification can include one or more physical additions to the surface of the one or more films 12, mesh spacers 14, and adhesion layers. The physical modifications can include fibers, bumps, ridges, pits, grooves, channels, and the like. The physical modifications can be introduced by any suitable method, including electrospinning, electrospraying, spin coating, dip coating, chemical vapor deposition, chemical solution deposition, physical vapor deposition, liquid bath immersion, thermal imprinting, engraving, stamping, microcontact printing, and the like.

In some embodiments, the one or more films 12, mesh spacers 14, and adhesion layers can be subject to one or more surface treatments. The application of the one or more surface treatments can introduce a texture, coating, or pattern to control the growth pattern of adhered cells. For example, the texture, coating, or pattern can promote or inhibit cell attachment, such that the cellular process, including differentiation, growth, migration, and proliferation can be controlled in a desirable manner.

In some embodiments, the one or more surface treatments can include one or more extracellular matrix material and/or blends of naturally occurring extracellular matrix material, including but not limited to collagen, fibrin, fibrinogen, thrombin, elastin, laminin, fibronectin, vitronectin, hyaluronic acid, chondroitin 4-sulfate, chondroitin 6-sulfate, dermatan sulfate, heparin sulfate, vixapatin (VP12), heparin, and keratan sulfate, proteoglycans, and combinations thereof. Some collagens that may be beneficial include but are not limited to collagen types I, II, III, IV, V, VI, VII, VIII, IX, X, XI, XII, XIII, XIV, XV, XVI, XVII, XVIII, and XIX. These proteins may be in any form, including but not limited to native and denatured forms. In various embodiments, the one or more surface treatments can include one or more carbohydrates such as chitin, chitosan, alginic acids, and alginates such as calcium alginate and sodium alginate. These materials may be isolated from plant products, humans or other organisms or cells or synthetically manufactured.

In various embodiments, the surface treatments can include natural peptides, such as glycyl-arginyl-glycyl-aspartyl-serine (GRGDS), arginylglycylaspartic acid (RGD), and amelogenin. In some embodiments, the surface treatments can include sucrose, fructose, cellulose, or mannitol. In some embodiments, the surface treatments can include nutrients, such as bovine serum albumin. In some embodiments, the surface treatments can include vitamins, such as vitamin B2, vitamin Ad, Vitamin D, Vitamin E, and Vitamin K. In some embodiments, the surface treatments can include nucleic acids, such as mRNA and DNA. In some embodiments, the surface treatments can include natural or synthetic steroids and hormones, such as dexamethasone, hydrocortisone, estrogens, and its derivatives. In some embodiments, the surface treatments can include growth factors, such as fibroblast growth factor (FGF), transforming growth factor beta (TGF-β), and epidermal growth factor (EGF). In some embodiments, the surface treatments can include a delivery vehicle, such as nanoparticles, microparticles, liposomes, viral and non-viral transfection systems.

In various embodiments, the surface treatments can include one or more therapeutics. The therapeutics can be natural or synthetic drugs, including but not limited to: analgesics, anesthetics, antifungals, antibiotics, anti-inflammatoirensteroidal anti-inflammatory drugs (NSAIDs), anthelmintics, antidotes, antiemetics, antihistamines, anti-cancer drugs, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents.

### Bioreactor Systems

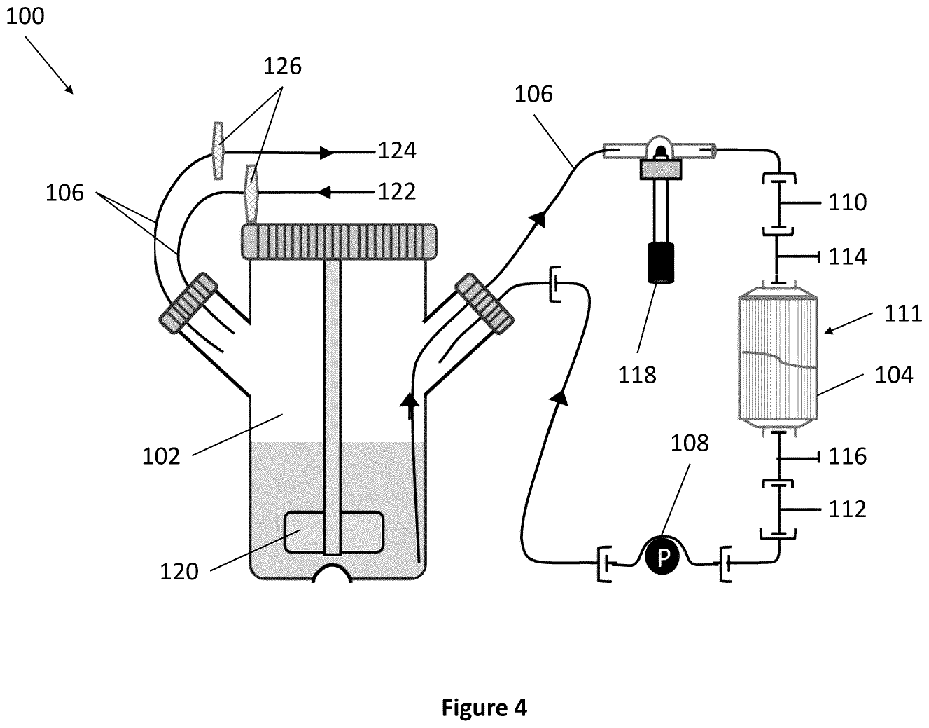

The present invention also relates to modular bioreactor systems for culturing and expanding cells using rolled scaffold devices. Referring now to FIG. 4, an exemplary modular bioreactor system 100 is depicted. Bioreactor system 100 comprises one or more reservoirs 102 connected by tubing 106 to one or more rolled scaffold devices 111, each device 111 being held in a cylindrical holder 104 having a hollow casing with one or more inlet ports at a first end and one or more outlet ports at an opposite second end. It should be understood that device 111 encompasses any rolled scaffold, including the mesh rolled scaffolds described herein as well as rolled scaffolds using spacers formed by molds or ultraviolet light curing (UV rolled scaffolds). As described in U.S. Provisional Application No. 62/621,635, UV rolled scaffolds comprise at least one substantially planar film having a plurality of elongate spacers attached to the film, wherein rolling the film causes the spacers to maintain a space between the rolled film layers. The space between the rolled film layers enable the UV rolled scaffolds to support a culture of adherent cells, such as within cylindrical holder 104 of bioreactor system 100.

The one or more devices 111, each within a cylindrical holder 104, can be connected to the one or more reservoirs 102 in series, in parallel, and combinations thereof. Pump 108 is connected to tubing 106 to power the circulation of media between the one or more reservoirs 102 and the one or more devices 111.

Each of the one or more reservoirs 102 can further comprise a stirring impeller 120 or stirrers for mixing purposes. Each of the one or more reservoirs 102 can further include a gas inlet 122 and a corresponding gas outlet 124 for gas exchange of culture medium, as well as a medium inlet/outlet for media perfusion (not pictured). The various inlets and outlets can include one or more filters 126 to preserve sterility or to remove unwanted particles. The reservoirs and other fluidic components can be pressurized between about 1 and 10 atm, and the reservoirs can be stirred at between about 100 and 10000 rpm for increased gas exchange rate.

Tubing 106 can include any number of flow diverting mechanisms, such as stopcocks, valves, and any other fluidic devices, such that the circulation of media can be directed in any desired fashion. For example, an upstream access port 114 can be used to introduce any desired content into the media circulation path. Exemplary content include cells, nucleic acid molecules, DNA, RNA, peptides, proteins, small molecules, dyes, hormones, vitamins, growth factors, stem cell factors, and the like. In another example, a downstream access port 116 can be used to capture samples of media flowing out of devices 111 or to collect harvested cells from devices 111.

Tubing 106 can further be adapted to include one or more probes and sensors to monitor the composition of the circulating fluids. For example, tubing 106 can further include an upstream sensor 110, a downstream sensor 112, and probe 118. The sensors and probes can include but are not limited to: temperature sensors, flow sensors, pH sensors, gas concentration sensors, analyte sensors, and the like.

### Dialysis Bioreactor System

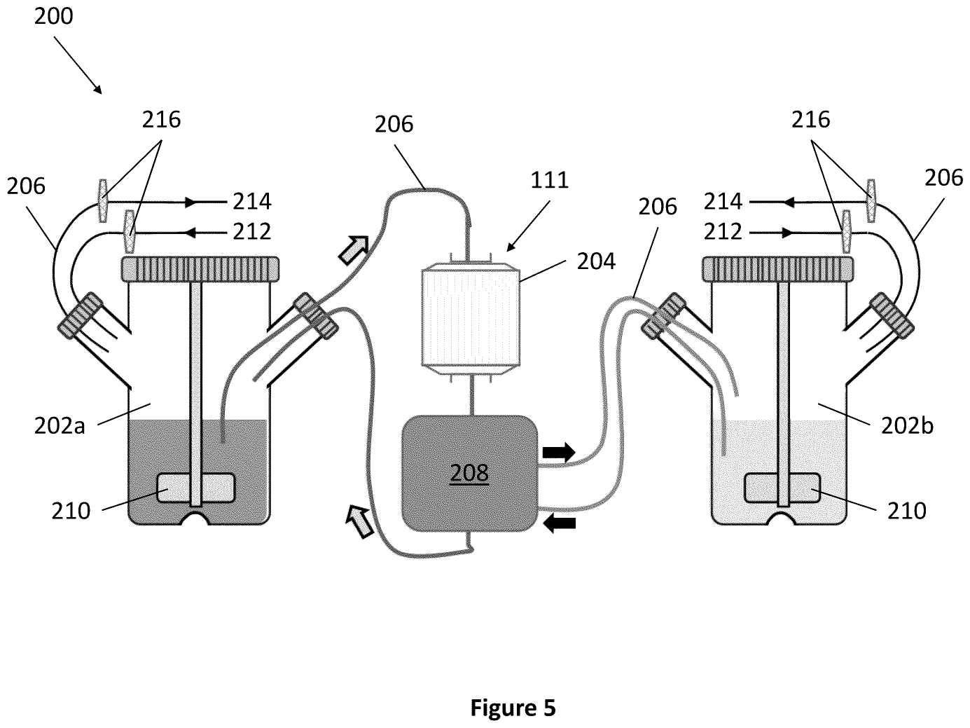

Referring now to FIG. 5, an exemplary bioreactor system 200 is depicted (pumps, sensors, and other components are omitted for clarity). Bioreactor system 200 is configured to separate components in a media stream, such as separating produced proteins and hormones from metabolic biowaste for ease of harvest and replacement. Bioreactor system 200 comprises at least a first reservoir 202a and a second reservoir 202b, each connected by tubing 206 to one or more rolled scaffold devices 111 (including mesh rolled scaffolds and UV rolled scaffolds) held in a cylindrical holder 204, and a dialyzer 208. In the depicted embodiment, first reservoir 202a contains culture media, and second reservoir 202b contains dialysate.

Dialyzer 208 is connected to tubing 206 downstream from cylindrical holder 204 to filter and exchange spent media from cylindrical holder 204. Dialyzer 208 can employ hollow fibers or membranes for removing components from the media flowing downstream from the one or more devices 111. In some embodiments, the components removed are metabolic biowastes, which can be diverted into second reservoir 202b or an additional reservoir. Dialyzer 208 can preserve certain components within a media stream and can separate out other components, including but not limited to as hormones, salts, antibodies, proteins, and the like. Separated components can be diverted into second reservoir 202b or other reservoir, which can be replaced. In some embodiments, molecules in the dialysate, such as glucose, amino acids, and other small molecular weight molecules, can be diffused to the media through the dialyzer.

Bioreactor system 200 can further include any desired modular bioreactor component, including but not limited to stirring impellers 210, gas inlets 212, gas outlets, 214, filters 216, stopcocks, valves, fluidic devices, sensors, and probes as described herein. It should be understood that the components of bioreactor system 200 are compatible with any of the modular bioreactor systems described herein.

### Temperature-Controlled Bioreactor System

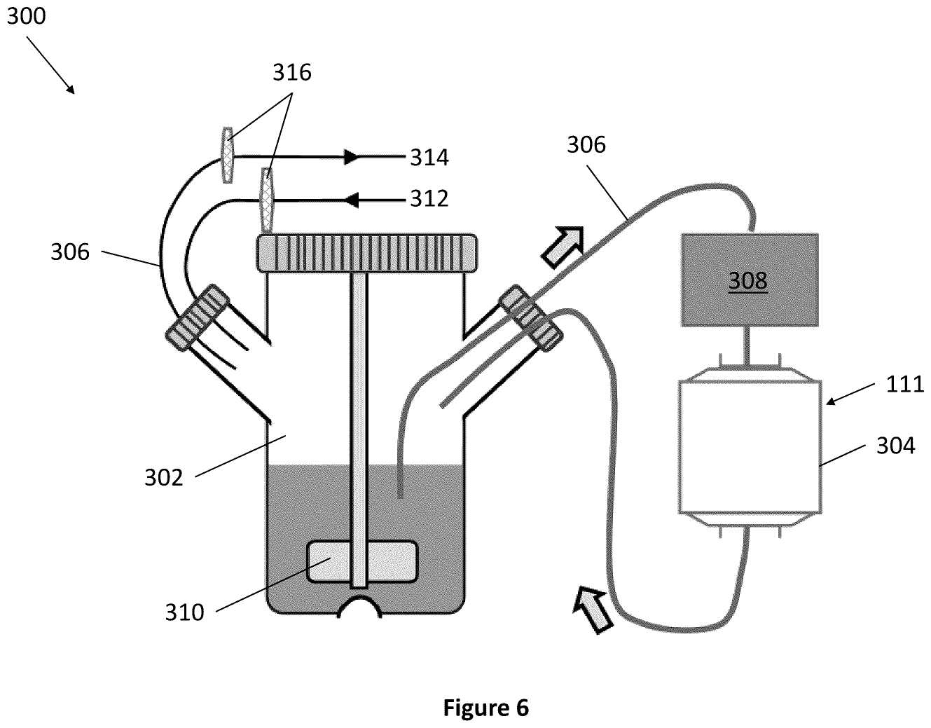

Referring now to FIG. 6, an exemplary bioreactor system 300 is depicted (pumps, sensors, and other components are omitted for clarity). Bioreactor system 300 comprises at least one reservoir 302 connected by tubing 306 to one or more rolled scaffold devices 111 (including mesh rolled scaffolds and UV rolled scaffolds) held in a cylindrical holder 304. Bioreactor system 300 further comprises heat exchanger 308 configured to change the temperature of the contents of tubing 306 outside of reservoir 302. In various embodiments, heat exchanger 308 can raise the temperature of the contents of tubing 306 or lower the contents of tubing 306.

For example, reservoir 302 can contain an amount of media that is optimally stored at a first temperature for enhanced stability and preservation. The amount of media is transported to cylindrical holder 304 by way of tubing 306, whereupon heat exchanger 308 changes the temperature of the amount of media to a second temperature before the amount of media contacts a culture of cells on the one or more devices 111. The second temperature can be a temperature that is physiologically optimal for the culture of cells, and is different from the first temperature. After passing through cylindrical holder 304, the media can be returned to reservoir 302 and incubated at the first temperature. In some embodiments, bioreactor system 300 can further comprise a second heat exchanger 308 downstream from cylindrical holder 304, wherein the second heat exchanger 308 can change the temperature of the amount of media leaving the cylindrical holder 304 to the first temperature, such that media returning to reservoir 302 does not significantly alter its incubating temperature.

Bioreactor system 300 can further include any desired modular bioreactor component, including but not limited to stirring impeller 310, gas inlets 312, gas outlets, 314, filters 316, stopcocks, valves, fluidic devices, sensors, probes, and bioreactor system 200 (depicted in FIG. 5) as described elsewhere herein. It should be understood that the components of bioreactor system 300 are compatible with any of the modular bioreactor systems described herein.

### Cell Culture

Cells can be cultured onto rolled scaffold devices 111 (including mesh rolled scaffolds and UV rolled scaffolds) prior to being connected to the bioreactor systems. Cells can also be introduced into the bioreactor systems, such as through one or more upstream access ports. The circulation of media can be temporarily halted, either by stopping the pumps or closing a stopcock or valve downstream from the cylindrical holders, to permit the cells to adhere to the devices 111. Once the cells have adhered, the pumps can be restarted or the stopcocks or valves can be reopened to restart circulation of media. The cells can be removed from the devices 111 by the application of any suitable cell dissociation solution. In some embodiments, the cells can be removed after removing the devices 111 from the bioreactor systems. In other embodiments, the cells can be removed by introducing a cell dissociation solution through the one or more upstream access ports. As described above, the circulation of media can be temporarily halted to permit the cell dissociation solution to detach the adhered cells from the devices 111 within the cylindrical holders. Once the cells have detached, circulation can be restarted, and the cells can be retrieved through the one or more downstream access ports.

The microenvironment is not affected by the increased capacity of culture. In suspension cultures with stirred bioreactors, increasing capacity leads to a decrease in the surface-to-volume ratio of the cell suspension and eventually leads to increased mechanical agitation and hydrodynamic shear stress. On the other hand, the culture capacity of the rolled scaffold is increased by increasing the number of identical channels without changing their geometry. Hence, the microenvironment of the rolled scaffold is independent of the culture capacity.

As a monolayer culture, it is easy to apply physical stimuli to cells. To mature cardiomyocytes and epithelial cells, electrical pulse and hydrodynamic shear stress are widely investigated. As the geometry of the rolled scaffolds is fully defined, it is straightforward to apply uniform electrical pulses via fixed electrodes.

Global metabolic activities of cells can be easily monitored in real time by comparing the measurements from the upstream and downstream sensors. The upstream access port can be used to inject cell suspensions for seeding and cell dissociation solution for harvesting, whereas the downstream access port is used to collect the harvested cells. The entire setup can be placed in an incubator, such as at 37° C. and 5% CO₂.

The medium or dialysate in the reservoir can be stirred and aerated vigorously without fear of damaging the cells, as the reservoirs and the mesh rolled scaffolds are separate. For example, the medium and dialysate can be stirred at rates between 100 rpm and 10000 rpm without harming the cells. Therefore, the mesh rolled scaffolds can support larger cell populations with lesser amounts of medium. Existing protocols for 2D culture can be easily adopted, as the cells grow in monolayers in the rolled scaffolds. The media in the mesh rolled scaffolds can be changed fast and efficiently while maintaining laminar flow with a low Reynold's number. This feature enhances transportation of gases and nutrients to cells, as well as preventing the build-up of metabolic byproducts and pH decreases, which can reduce cell proliferation and pluripotency. Gas exchange can be further enhanced by placing the bioreactor system or at least one reservoir in a controlled environment having a specified temperature, humidity, pressure, or gas content. In some embodiments, the bioreactor system or portions of the bioreactor system can be placed within a pressurized chamber at between about 1 and 10 atm. Rapid exchange of media in the mesh rolled scaffolds also facilitates seeding and harvesting.

The cells that can be cultured using the rolled scaffolds of the present invention can be any suitable cell. For example, in some embodiments the cells can include progenitor cells, pluripotent cells, stem cells, other differentiable cells, and the like. In some embodiments, the rolled scaffolds of the present invention direct differentiation of progenitor cells and/or stem cells. In some embodiments, the rolled scaffolds of the present invention direct and maintain phenotype plasticity of the cells that are seeded therein. In some embodiments, the rolled scaffolds of the present invention are used to support niche expansion of stem cells seeded therein. In some embodiments, the rolled scaffolds of the present invention can be used to culture recombinant cells to produce biopharmaceutical products, including therapeutic proteins and monoclonal antibodies.

In some embodiments, the compositions and methods useful with the present invention enhance the culturing of cells, for example, differentiable cells such as induced pluripotent stem cells, embryonic stems cells, hematopoietic stem cells, adipose derived stem cells, bone marrow derived stem cells and the like. In some embodiments, the differentiatable cells are directed to differentiate into cells of target tissues, for example fibroblasts, osteocytes, epithelial cells, cardiomyocytes, endothelial cells, myocytes, neurocytes, and the like. In some embodiments, at different points during culturing the differentiable cells, various components may be added to the cell culture such that the medium can contain components such as growth factors, differentiation factors, and the like other than those described herein.

In some embodiments, the compositions and methods can comprise a basal salt nutrient solution. A basal salt nutrient solution refers to a mixture of salts that provide cells with water and certain bulk inorganic ions essential for normal cell metabolism, maintain intra- and extra-cellular osmotic balance, provide a carbohydrate as an energy source, and provide a buffering system to maintain the medium within the physiological pH range. For example, basal salt nutrient solutions may include, but are not limited to, Dulbecco's Modified Eagle's Medium (DMEM), Minimal Essential Medium (MEM), Basal Medium Eagle (BME), RPM1 1640, Hams F-10, Ham's F-12, β-Minimal Essential Medium (β-MEM), Glasgow's Minimal Essential Medium (G-MEM), and Iscove's Modified Dulbecco's Medium, and mixtures thereof. In some embodiments, the basal salt nutrient solution is an approximately 50:50 mixture of DMEM and Ham's F12.

In some embodiments, the compositions and methods useful with the present invention provide for one or more soluble attachment factors or agents, such as soluble attachment components as contained in the human serum, which at the appropriate concentration range facilitates cell attachment to tissue culture type plastic and or the surface of the rolled scaffold. Such cell attachment allows cells to attach and form a monolayer but in the absence of a feeder layer or a substrate coating, e.g., a matrix coating, Matrigel, and the like. In some embodiments, human serum is utilized in order to provide an animal-free environment. In some embodiments, serum from animal sources, for example goat, calf, bovine, horse, mouse, and the like is utilized. Serum can be obtained from any commercial supplier of tissue culture products, examples include Gibco-Invitrogen Corporation (Grand Island, N.Y. USA), Sigma (St. Louis Mo., USA) and the ATCC (Manassas, Va. USA). The serum used may be provided at a concentration range of about 0.1% to about 20%, about 5% to about 15%, about 7% to about 12%, about 10%, 0.1 to about 3%, about 0.5 to about 2%, about 0.5 to about 1.5%, and about 0.5 to about 1%.

In some embodiments, as contemplated herein, the cells on the rolled scaffolds can be passaged using enzymatic, non-enzymatic, or manual dissociation methods prior to and/or after contact with a defined medium. Non-limiting examples of enzymatic dissociation methods include the use of proteases such as trypsin, collagenase, dispase, and accutase (marine-origin enzyme with proteolytic and collagenolytic enzymes in phosphate buffered saline; Life Technologies, Carlsbad, Calif.). In some embodiments, accutase is used to passage the contacted cells. When enzymatic passaging methods are used, the resultant culture can comprise a mixture of singlets, doublets, triplets, and clumps of cells that vary in size depending on the enzyme used. A non-limiting example of a non-enzymatic dissociation method is a cell dispersal buffer. Manual passaging techniques have been well described in the art, such as in Schulz et al., 2004 Stem Cells, 22(7):1218-38. The choice of passaging method is influenced by other culture conditions, including but not limited to feeders and/or extracellular matrices.

In some embodiments, the methods described herein allow for expansion of cells, followed by detaching the cells from the mesh rolled scaffolds and passaging of the detached cells on the mesh rolled scaffolds or similar cell culture devices, so that the cells retain their characteristics such as pluripotency through expansion and serial passages. In addition, the methods of expansion and passage described herein are carried out in a closed system which ensures sterility during the production process.

Methods of inducing differentiation are known in the art and can be employed to induce the desired stem cells to give rise to cells having a mesodermal, ectodermal or endodermal lineage.

After culturing the stem cells in a differentiating-inducing medium for a suitable time (e.g., several days to a week or more), the stem cells can be assayed to determine whether, in fact, they have acquired the desired lineage.

Methods to characterize differentiated cells that develop from the stem cells of the invention, include, but are not limited to, histological, morphological, biochemical and immunohistochemical methods, or using cell surface markers, or genetically or molecularly, or by identifying factors secreted by the differentiated cell, and by the inductive qualities of the differentiated stem cells.

In another embodiment, the cells can be genetically modified, e.g., to express exogenous (e.g., introduced) genes (“transgenes”) or to repress the expression of endogenous genes, and the invention provides a method of genetically modifying such cells and populations. In accordance with this method, the cells are exposed to a gene transfer vector comprising a nucleic acid including a transgene, such that the nucleic acid is introduced into the cell under conditions appropriate for the transgene to be expressed within the cell. The transgene generally is an expression cassette, including a polynucleotide operably linked to a suitable promoter. The polynucleotide can encode a protein, or it can encode biologically active RNA (e.g., antisense RNA or a ribozyme).

The expression cassette containing the transgene should be incorporated into a genetic vector suitable for delivering the transgene to the cells. Depending on the desired end application, any such vector can be so employed to genetically modify the cells (e.g., plasmids, naked DNA, viruses such as adenovirus, adeno-associated virus, herpesviruses, lentiviruses, papillomaviruses, retroviruses, etc.). Any method of constructing the desired expression cassette within such vectors can be employed, many of which are well known in the art (e.g., direct cloning, homologous recombination, etc.). Of course, the choice of vector will largely determine the method used to introduce the vector into the cells (e.g., by protoplast fusion, calcium-phosphate precipitation, gene gun, electroporation, infection with viral vectors, etc.), which are generally known in the art.

The genetically altered cells can be employed to produce the product of the transgene. In other embodiments, the genetically modified cells are employed to deliver the transgene and its product to an animal. For example, the cells, once genetically modified, can be introduced into the animal under conditions sufficient for the transgene to be expressed in vivo.

In other embodiments, cells can be employed as therapeutic agents, for example in cell therapy applications. Generally, such methods involve transferring the cells to desired tissue, either in vitro (e.g., as a graft prior to implantation or engrafting) or in vivo, to animal tissue directly. The cells can be transferred to the desired tissue by any method appropriate, which generally will vary according to the tissue type. For example, cells can be transferred to a graft by bathing the graft (or infusing it) with culture medium containing the cells. Alternatively, the cells can be seeded onto the desired site within the tissue to establish a population. Cells can be transferred to sites in vivo using devices such as catheters, trocars, cannulae, stents (which can be seeded with the cells), etc.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1: Mesh Rolled Scaffold Platform for Large-Scale Cell Culture

The following study demonstrates mesh rolled scaffolds as a viable platform for large-scale cell culture. The expansion of Chinese hamster ovary (CHO) cells were demonstrated.

### Fabrication and Demonstration of the Rolled Scaffold

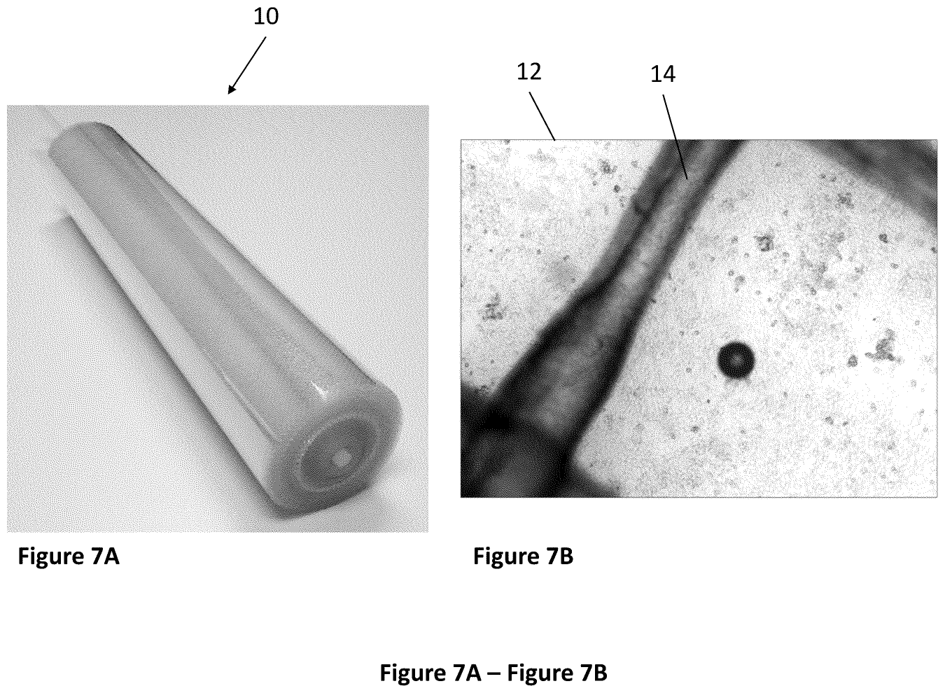

Exemplary dimensions of mesh rolled scaffolds are presented in Table 1. FIG. 7A shows a fabricated mesh rolled scaffold before it is encapsulated in a rolled scaffold holder. The mesh rolled scaffold is placed in to a cylindrical holder that has inlet and outlet fluidic ports on opposite ends to allow media flow. The rolled scaffold in this example is a 50 μm polyethylene terephthalate (PET) film and a plastic netting (Naltex, Delstar, Inc., USA) as a feed spacer.

TABLE 1
Exemplary mesh rolled scaffold sizes and dimensions.

| | Small | Medium | Large |
|---|---|---|---|
| Length (cm) | 10 | 15 | 30 |
| Radius (mm) | 7.25 | 10.75 | 28 |
| Available Area (cm²) | 350 | 1576 | 22000 |
| Volume (mL) | 16 | 54.5 | 738.8 |
| Area to Volume Ratio (cm²/mL) | 21.875 | 28.9174 | 29.778 |

### Cell Culture Setup

Small and medium mesh rolled scaffolds were tested with the experimental setup shown in FIG. 4. The culture medium is oxygenated in a spinner flask and pumped into the mesh rolled scaffold with a peristatic pump. The cells in the mesh rolled scaffold consume oxygen and nutrients in the medium as it flows through the mesh rolled scaffold. Oxygen concentration is measured at upstream and downstream of the mesh rolled scaffold to measure the combined oxygen consumption rate of the cells, which is proportional to the total number of the cells and their metabolic level. pH and glucose concentration levels of the media in the spinner flask are also routinely measured. The biocompatibility of the mesh rolled scaffolds with the culture of CHO cells was demonstrated, as shown in FIG. 7B.

### Results

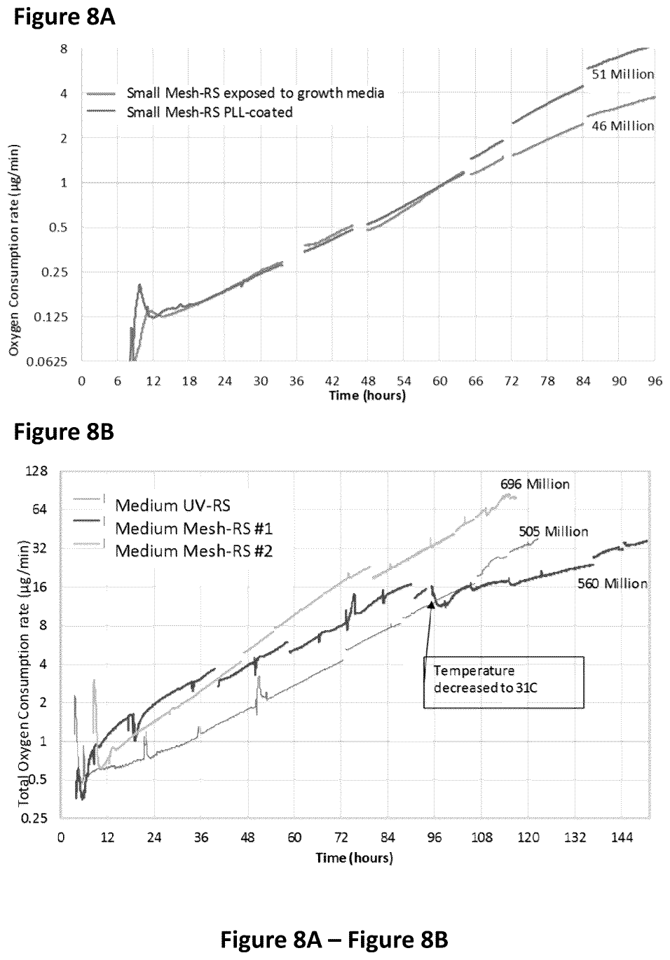

FIG. 8A shows the oxygen consumption rate of the cells growing in a small mesh rolled scaffold that was coated with poly-L-lysine (PLL) and one that was exposed to growth media before seeding. In both cases, the oxygen consumption rate and cell number increased exponentially. 51 million and 46 million CHO cells were harvested from PLL-coated small mesh rolled scaffold and growth-media exposed small mesh rolled scaffold, respectively. Both the results show a doubling time of 20 hours and 20.7 hours.

FIG. 8B shows the oxygen consumption rate of the cells growing in the medium mesh rolled scaffold that is exposed to growth media before seeding. As shown in the graph, mesh rolled scaffolds can support the growth of CHO cells equal to or better than rolled scaffolds with spacers. For medium mesh rolled scaffold #1, the culture temperature was decreased from 37° C. to 31° C. to simulate the production phase of therapeutic proteins. Although the growth rate of CHO cells were significantly decreased, the combined metabolism was maintained, showing viability of CHO cells at a decreased temperature.

### Example 2: Rolled Scaffold Dialysis Bioreactor for Biopharmaceutical Production

In biopharmaceutical industries, recombinant cells are used to produce therapeutic proteins and monoclonal antibodies for advanced diagnostics and cancer treatment. In these applications, animal cells are genetically modified to secrete specific target molecules and cultured in a large-scale bioreactor with suspension culture. As the cells produce the target molecules, they also consume key nutrients and generate metabolic biowaste, such as lactate and ammonia. Currently, two strategies are used to address the nutrient depletion and the accumulation of metabolic biowaste in culture media, fed-batch mode, or perfusion mode. In the fed-batch mode, concentrated nutrients or additional culture media are added to the bioreactor when nutrients are depleted or metabolic biowaste is accumulated. The perfusion mode continuously and simultaneously adds fresh media and removes the same volume of used media in the bioreactor, allowing removal of waste, supply of nutrients, and harvesting of product. To prevent accumulation of the metabolic biowaste, additional culture medium is added to the bioreactor to dilute the metabolic waste (fed-batch mode) or the culture medium in the bioreactor is continuously replaced with fresh culture medium (perfusion mode). Both of these approaches dilute or remove the produced target protein in the bioreactor, making it difficult to produce a very high concentration of the target protein. The fundamental reason behind this is that the target protein and the biowaste cannot be separated or treated in a different manner with the fed-batch or the perfusion mode.

In the present study, the rolled scaffold dialysis (RSD) bioreactor shown in FIG. 5 can selectively remove the metabolic biowaste and keep the produced target protein in the bioreactor. Most of the produced target proteins have molecular weights (100-140 kDa) much higher than those of the metabolic biowastes (less than 1 kDa). By setting the molecular weight cut off (MWCO) of the dialysis membrane in the range of 10-50 kDa, the produced protein or antibody will remain in the media reservoir, while metabolic biowaste is removed through dialysis. At the same time, by using basal media with low molecular weight (MW) nutrients as the dialysate, the depleted nutrients with low MW, such as glucose and amino acids can be also replenished through the dialysis membrane. The dialysate will be replaced in a timely manner for proper operation of dialysis. Unlike earlier reports, where the metabolic biowaste is immediately diluted in media reservoir, the unique configuration of the RSD bioreactor allows efficient removal of the metabolic biowaste before it is diluted in the media reservoir. This approach is possible, only with the use of rolled scaffolds, which inherently separates the cells from the main media reservoir.

The RSD bioreactor accumulated the produced target protein in the media reservoir without loss and increased the concentration of the target protein significantly. The biowaste was removed through a dialysis filter and the low molecular weight nutrients can be replenished continuously, which extends the production phase. Combined together, these improvements significantly enhance the titer in the biopharmaceutics production and reduce the manufacturing cost of the biopharmaceutics.

### Example 3: Rolled Scaffold Low-Temperature Media Bioreactor

The high production cost of stem cells is one of the major technical hurdles in stem cell therapy, regenerative medicine, and advanced tissue engineering. The large consumption of expensive culture medium is one of the major cost factors of stem cell production. In the present study, a rolled scaffold low-temperature media (LTM) bioreactor is demonstrated, which significantly reduces the usage time of costly culture medium.

The typical composition of the culture media used for induced pluripotent stem cell (iPSC) expansion contains basal media such as DMEM, low molecular weight compounds, and recombinant proteins for appropriate cell signaling, as shown in Table 2. Among these elements, recombinant proteins are most expensive and take up more than 96% of the total cost for the culture media. Unfortunately, fibroblast growth factor 2 (FGF2) in Table 3, which promotes survival and proliferation of stem cells, is thermally unstable at 37° C. and degrades significantly in 24 hours. For this reason, the culture media should be exchanged daily, increasing the usage of the expensive culture media and the manufacturing cost of the stem cells.

TABLE 2
The composition of E8 media for stem cell culture. The majority
of the media cost (96%) is generated from 4 recombinant
proteins with MW over 5.8 kDa. The retail costs are from
Thermo-Fisher (PN: 11965-084, DMEM/F12) and Sigma Aldrich
(PN: A5960, S5261, S5761, T3309, I3536, F0291, T7039).

| | Molecular weight & | |
|---|---|---|
| Components | concentration | Cost factor (%) |
| DMEM/F12 | | 3.7% |
| L-ascorbic acid | 176.12 Da | 0% |
| Sodium Selenite | 172.94 Da | 0% |
| NaHCO₃ | 84.01 Da | 0% |
| Transferrin (human) | 76-81 kDa, 10.7 mg/L | 0.7% |
| Insulin (human) | 5.8 kDa, 19.4 mg/L | 10.5% |
| FGF2 (human) | 16-17 kDa, 100 μg/L | 81.7% |
| TGFβ1 (human) | 25 kDa, 2 μg/L | 3% |

To address this issue, the majority of the culture media can be kept at low temperature (4° C.) to extend the longevity of the recombinant protein and the culture media. With the use of rolled scaffolds, the cells are separated from the media reservoir. The media reservoir can be kept at low temperature for enhanced longevity of FGF2 and the media can be heated with a heat exchanger immediately before entering the rolled scaffold at physiological temperature (37° C.), as shown in FIG. 6.

By storing the culture media with temperature-sensitive proteins at low temperature, the expensive culture media can be used for a longer period and the manufacturing cost of stem cells can be significantly reduced. If needed, the setup can be combined with a rolled scaffold dialysis bioreactor so that the biowaste with low molecular weight can be readily removed and that the small molecule nutrients can be continuously replenished.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1-15. (canceled)

16. A bioreactor system, comprising:
a rolled scaffold;
at least one cylindrical holder, each comprising a hollow casing sized to fit a rolled scaffold, at least one inlet port at a first end, and at least one outlet port at an opposite second end;
at least one reservoir;
tubing fluidically connecting the at least one reservoir to each of the cylindrical holders; and
at least one pump connected to the tubing.

17. The bioreactor system of claim 16, wherein the rolled scaffold is constructed from at least one substantially planar film and at least one mesh netting rolled into a cylindrical rolled scaffold having alternating layers of film and mesh netting.

18. The bioreactor system of claim 16, wherein the rolled scaffold is constructed from at least one substantially planar film, at least one mesh netting, and at least one adhesion layer rolled into a cylindrical rolled scaffold having alternating layers of film, mesh netting, and adhesion layers.

19. The bioreactor system of claim 16, wherein the rolled scaffold is constructed from at least one substantially planar film having a plurality of elongate spacers attached to the film rolled into a cylindrical rolled scaffold, such that the spacers maintain a space between the rolled film layers.

20. The bioreactor system of claim 16, wherein the at least one reservoir is fluidically connected to one or more media sources, gas sources, chemical reagents, and combinations thereof.

21. The bioreactor system of claim 16, wherein the tubing comprises one or more access ports upstream from the cylindrical holders, downstream from the cylindrical holders, or both.

22. The bioreactor system of claim 16, wherein the tubing comprises one or more sensors upstream from the cylindrical holders, downstream from the cylindrical holders, or both.

23. The bioreactor system of claim 21, wherein the one or more sensors are selected from the group consisting of: temperature sensors, flow sensors, pH sensors, gas concentration sensors, glucose sensors, and analyte sensors.

24. The bioreactor system of claim 16, wherein the tubing comprises one or more stopcocks or valves configured to stop or divert flow of fluid within the system.

25. The bioreactor system of claim 16, wherein the rolled scaffold, each within a cylindrical holder, is connected to the at least one reservoir in series, in parallel, and combinations thereof.

26. The bioreactor system of claim 16, wherein the tubing further comprises a dialyzer configured to separate out components of fluid within the tubing and to introduce components into fluid within the tubing.

27. The bioreactor system of claim 16, wherein the tubing further comprises at least one heat exchanger configured to change the temperature of fluid within the tubing, such that the at least one reservoir is maintained at a temperature that is different from the temperature of the rolled scaffold.

28. The bioreactor system of claim 26, wherein the at least one heat exchanger is positioned upstream from the rolled scaffold, downstream from the rolled scaffold, or both.

29. The bioreactor system of claim 16, wherein the at least one reservoir comprises a stirring impeller configured to rotate between 100 and 10000 rpm.

30. The bioreactor system of claim 16, wherein the system is pressurized between 1 atm and 10 atm.

31. The bioreactor system of claim 16, wherein the tubing further comprises at least one dialyzer and at least one heat exchanger.
